(19) United States
(12) Patent Application Publication
(10) Pub. No.: US 2009/0176680 A1
(43) Pub. Date: Jul. 9, 2009

(54) USE OF A CELLULASE TO IMPART SOIL RELEASE BENEFITS TO COTTON DURING A SUBSEQUENT LAUNDERING PROCESS

(76) Inventors: Steven George Patterson, Tyne & Wear (GB); Neil Joseph Lant, Newcastle (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/341,740

(22) Filed: Dec. 22, 2008

Related U.S. Application Data
(60) Provisional application No. 61/010,111, filed on Jan. 4, 2008.
(60) Provisional application No. 61/114,568, filed on Nov. 14, 2008.

(51) Int. Cl. C11D 3/386; C12N 9/24

(52) U.S. Cl. 510/300; 435/200

(57) ABSTRACT

The present invention relates to the use of a cellulase to impart soil release benefits to cotton during a subsequent laundering process.

### CROSS REFERENCE TO RELATED APPLICATIONS

This application claims the benefit of U.S. Provisional Application No. 61/010,111 filed 4 Jan. 2008; and U.S. Provisional Application No. 61/114,568 filed 14 Nov. 2008.

### FIELD OF THE INVENTION

The present invention relates to the use of a cellulase to impart soil release benefits to cotton during a subsequent laundering process.

### BACKGROUND OF THE INVENTION

Cellulases have been incorporated into laundry detergent compositions to impart de-pilling, fabric-softness, colour clarification, particulate soil removal, anti-redeposition and cereal soil removal benefits. However, the Inventors have identified another surprising benefit of cellulases, namely their use to impart soil release benefits to cotton during a laundering process. Without wishing to be bound by theory, the Inventors believe that the cellulases increase the micro-porosity of the cotton fibres during the laundering process leading to improved removal of soils adhered to the fabric after the laundering process during wearing and usage of the fabric, in subsequent wash cycles. This durable, subsequent wash benefit is known as soil release. Whilst cellulases were known to impart a first wash soil removal benefit, they were not known to impart soil release benefits during the second and third wash cycles.

### SUMMARY OF THE INVENTION

The present invention relates to the use of a cellulase to impart soil release benefits to cotton during a subsequent laundering process. The soil release benefit is observed on cotton fabric and on all types of fabric that comprise a significant amount of cotton, such as cotton-synthetic (e.g. polyester, polyamide such as Nylon™, and elastane) blends.

### DETAILED DESCRIPTION OF THE INVENTION

### Cellulase

The cellulase is an endoglucanase. Preferably, the cellulase has endo beta 1,4-glucanase activity and a structure which does not comprise a class A Carbohydrate Binding Module (CBM). A class A CBM is defined according to A. B. Boraston et al. Biochemical Journal 2004, Volume 382 (part 3) pages 769-781. In particular, the cellulase does not comprise a class A CBM from families 1, 2a, 3, 5 and 10.

The cellulase preferably is a glycosyl hydrolase having enzymatic activity towards amorphous cellulose substrates, wherein the glycosyl hydrolase is selected from GH families 5, 7, 12, 16, 44 or 74. Preferably, the cellulase is a glycosyl hydrolase is selected from GH family 5. A preferred cellulase is Celluclean, supplied by Novozymes. This preferred cellulase is described in more detail in WO2002/099091. The glycosyl hydrolase (GH) family definition is described in more detail in Biochem J. 1991, v280, 309-316.

Another preferred cellulase is a glycosyl hydrolase having enzymatic activity towards both xyloglucan and amorphous cellulose substrates, wherein the glycosyl hydrolase is selected from GH families 5, 12, 44 or 74. Preferably, the glycosyl hydrolase selected from GH family 44.

The enzymatic activity towards xyloglucan substrates is described in more detail below. The enzymatic activity towards amorphous cellulose substrates is described in more detail below.

The glycosyl hydrolase enzyme preferably belongs to glycosyl hydrolase family 44.

The glycosyl hydrolase enzyme preferably has a sequence at least 70%, or at least 75% or at least 80%, or at least 85%, or at least 90%, or at least 95% identical to sequence ID No. 1.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows: (Identical Residues×100)/(Length of Alignment−Total Number of Gaps in Alignment).

Suitable glycosyl hydrolases are selected from the group consisting of: GH family 44 glycosyl hydrolases from Paenibacillus polyxyma (wild-type) such as XYG1006 described in WO 01/062903 or are variants thereof; GH family 12 glycosyl hydrolases from Bacillus licheniformis (wild-type) such as Seq. No. ID: 1 described in WO 99/02663 or are variants thereof; GH family 5 glycosyl hydrolases from Bacillus agaradhaerens (wild type) or variants thereof; GH family 5 glycosyl hydrolases from Paenibacillus (wild type) such as XYG1034 and XYG 1022 described in WO 01/064853 or variants thereof; GH family 74 glycosyl hydrolases from Jonesia sp. (wild type) such as XYG 1020 described in WO 2002/077242 or variants thereof; and GH family 74 glycosyl hydrolases from Trichoderma Reesei (wild type), such as the enzyme described in more detail in Sequence ID no. 2 of WO03/089598, or variants thereof.

Preferred glycosyl hydrolases are selected from the group consisting of: GH family 44 glycosyl hydrolases from Paenibacillus polyxyma (wild-type) such as XYG1006 or are variants thereof.

Typically, the cellulase modifies the fabric surface during the laundering process so as to improve the removal of soils adhered to the fabric after the laundering process during wearing and usage of the fabric, in subsequent wash cycles. Preferably, the cellulase modifies the fabric surface during the laundering process so as to improve the removal of soils adhered to the fabric after the laundering process during wearing and usage of the fabric, in the subsequent two, or even three wash cycles.

Typically, the cellulase is used at a concentration of 0.005 ppm to 1.0 ppm in the wash liquor during the first laundering process. Preferably, the cellulase is used at a concentration of 0.02 ppm to 0.5 ppm in the wash liquor during the first laundering process.

### Enzymatic Activity Towards Xyloglucan Substrates

An enzyme is deemed to have activity towards xyloglucan if the pure enzyme has a specific activity of greater than 50000 XyloU/g according to the following assay at pH 7.5.

The xyloglucanase activity is measured using AZCL-xyloglucan from Megazyme, Ireland as substrate (blue substrate).

A solution of 0.2% of the blue substrate is suspended in a 0.1 M phosphate buffer pH 7.5, 20° C. under stirring in a 1.5 ml Eppendorf tubes (0.75 ml to each), 50 microlitres enzyme solution is added and they are incubated in an Eppendorf Thermomixer for 20 minutes at 40° C., with a mixing of 1200 rpm. After incubation the coloured solution is separated from the solid by 4 minutes centrifugation at 14,000 rpm and the absorbance of the supernatant is measured at 600 nm in a 1 cm cuvette using a spectrophotometer. One XyloU unit is defined as the amount of enzyme resulting in an absorbance of 0.24 in a 1 cm cuvette at 600 nm.

Only absorbance values between 0.1 and 0.8 are used to calculate the XyloU activity. If an absorbance value is measured outside this range, optimization of the starting enzyme concentration should be carried out accordingly.

### Enzymatic Activity Towards Amorphous Cellulose Substrates

An enzyme is deemed to have activity towards amorphous cellulose if the pure enzyme has a specific activity of greater than 20000 EBG/g according to the following assay at pH 7.5. Chemicals used as buffers and substrates were commercial products of at least reagent grade.

### Endoglucanase Activity Assay Materials:

0.1M phosphate buffer pH 7.5
Cellazyme C tablets, supplied by Megazyme International, Ireland.
Glass microfiber filters, GF/C, 9cm diameter, supplied by Whatman.

### Method:

In test tubes, mix 1 ml pH 7,5 buffer and 5 ml deionised water.
Add 100 microliter of the enzyme sample (or of dilutions of the enzyme sample with known weight:weight dilution factor). Add 1 Cellazyme C tablet into each tube, cap the tubes and mix on a vortex mixer for 10 seconds. Place the tubes in a thermostated water bath, temperature 40° C.
After 15, 30 and 45 minutes, mix the contents of the tubes by inverting the tubes, and replace in the water bath. After 60 minutes, mix the contents of the tubes by inversion and then filter through a GF/C filter. Collect the filtrate in a clean tube.
Measure Absorbance (Aenz) at 590 nm, with a spectrophotometer. A blank value, Awater, is determined by adding 100 μl water instead of 100 microliter enzyme dilution.
Calculate Adelta=Aenz−Awater.
Adelta must be <0.5. If higher results are obtained, repeat with a different enzyme dilution factor.
Determine DFO.1, where DFO.1 is the dilution factor needed to give Adelta=0.1.
Unit Definition: 1 Endo-Beta-Glucanase activity unit (1 EBG) is the amount of enzyme that gives Adelta=0.10, under the assay conditions specified above. Thus, for example, if a given enzyme sample, after dilution by a dilution factor of 100, gives Adelta=0.10, then the enzyme sample has an activity of 100 EBG/g.

### Laundry Detergent Composition

The cellulase is preferably incorporated into a laundry detergent composition. The composition typically comprises detersive surfactants, typically anionic detersive surfactants. The composition may also comprise soil release polymers, preferably cellulosic polymers. Preferred cellulosic polymers are described in more detail below. Other suitable soil release polymers are described in WO01/62885. Other suitable soil release polymers are polyester soil release polymers. Other suitable soil release polymers are Repel-o-tex polymers, including Repel-o-tex SF, SF-2 and SRP6 supplied by Rhodia. Other preferred soil release polymers Texcare polymers, including Texcare SRA100, SRA300, SRN100, SRN170, SRN240, SRN300 and SRN325 supplied by Clariant. Other preferred soil release polymers are Marloquest polymers, such as Marloquest SL supplied by Sasol.

The laundry detergent composition can be in any form, such as a solid, liquid, gel or any combination thereof. The composition may be in the form of a tablet or pouch, including multi-compartment pouches. The composition can be in the form of a free-flowing powder, such as an agglomerate, spray-dried powder, encapsulate, extrudate, needle, noodle, flake, or any combination thereof. However, the composition is preferably in the form of a liquid. Additionally, the composition is in either isotropic or anisotropic form. Preferably, the composition, or at least part thereof, is in a lamellar phase.

The composition preferably comprises low levels of water, such as from 0.01 wt % to 5 wt %, preferably to 4 wt %, or to 3 wt %, or to 2 wt %, or even to 1 wt %. This is especially preferred if the composition is in the form of a pouch, typically being at least partially, preferably completely enclosed by a water-soluble film. The water-soluble film preferably comprises polyvinyl alcohol.

Solid Laundry Detergent Composition

In one embodiment of the present invention, the composition is a solid laundry detergent composition, preferably a solid laundry powder detergent composition.

The composition preferably comprises from 0 wt % to 10 wt %, or even to 5 wt % zeolite builder. The composition also preferably comprises from 0 wt % to 10 wt %, or even to 5 wt % phosphate builder.

The composition typically comprises anionic detersive surfactant, preferably linear alkyl benzene sulphonate, preferably in combination with a co-surfactant. Preferred co-surfactants are alkyl ethoxylated sulphates having an average degree of ethoxylation of from 1 to 10, preferably from 1 to 3, and/or ethoxylated alcohols having an average degree of ethoxylation of from 1 to 10, preferably from 3 to 7.

The composition preferably comprises chelant, preferably the composition comprises from 0.3 wt % to 2.0 wt % chelant. A suitable chelant is ethylenediamine-N,N′-disuccinic acid (EDDS).

The composition may comprise cellulose polymers, such as sodium or potassium salts of carboxymethyl cellulose, carboxyethyl cellulose, sulfoethyl cellulose, sulfopropyl cellulose, cellulose sulfate, phosphorylated cellulose, carboxymethyl hydroxyethyl cellulose, carboxymethyl hydroxypropyl cellulose, sulfoethyl hydroxyethyl cellulose, sulfoethyl hydroxypropyl cellulose, carboxymethyl methyl hydroxyethyl cellulose, carboxymethyl methyl cellulose, sulfoethyl methyl hydroxyethyl cellulose, sulfoethyl methyl cellulose, carboxymethyl ethyl hydroxyethyl cellulose, carboxymethyl ethyl cellulose, sulfoethyl ethyl hydroxyethyl cellulose, sulfoethyl ethyl cellulose, carboxymethyl methyl hydroxypropyl cellulose, sulfoethyl methyl hydroxypropyl cellulose, carboxymethyl dodecyl cellulose, carboxymethyl dodecoyl cellulose, carboxymethyl cyanoethyl cellulose, and sulfoethyl cyanoethyl cellulose. The cellulose may be a substituted cellulose substituted by two or more different substituents, such as methyl and hydroxyethyl cellulose.

The composition may comprise soil release polymers, such as Repel-o-Tex™. Other suitable soil release polymers are anionic soil release polymers. Suitable soil release polymers are described in more detail in WO05123835A1, WO07079850A1 and WO08110318A2.

The composition may comprise a spray-dried powder. The spray-dried powder may comprise a silicate salt, such as sodium silicate.

### Detersive Surfactant

The composition comprises detersive surfactant. The detersive surfactant The compositions preferably comprise from 2% to 50% surfactant, more preferably from 5% to 30%, most preferably from 7% to 20% detersive surfactant. The composition may comprise from 2% to 6% detersive surfactant. The detersive surfactant can be anionic, non-ionic, cationic or zwitterionic. Preferably, the detersive surfactant is anionic. The composition preferably comprises detersive surfactant in an amount to provide from 100 ppm to 5,000 ppm detersive surfactant in the wash liquor during the laundering process. This is especially preferred when from 10 g to 125 g of liquid laundry detergent composition is dosed into the wash liquor during the laundering process. The composition upon contact with water typically forms a wash liquor comprising from 0.5 g/l to 10 g/l detergent composition.

### Cellulosic Polymer

The cellulosic polymer is typically a cellulose or a modified cellulose. Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Other suitable cellulosic polymers include cationic cellulose and derivatives thereof. Suitable cationic cellulose is available from Amerchol Corp. (Edison, N.J., USA) in their Polymer JR™ and LR™ series of polymers. Other suitable cationic cellulose is the form of a salt of hydroxyethyl cellulose that is reacted with trimethyl ammonium substituted epoxide, such as that supplied by Amerchol Corp. under the tradename Polyquaternium 10™. Another suitable type of cationic cellulose includes the polymeric quaternary ammonium salts of hydroxyethyl cellulose reacted with lauryl dimethyl ammonium-substituted epoxide, such as that supplied by Amerchol Corp. under the tradename Polyquaternium 24™. Suitable cellulosic polymers are supplied by Amerchol Corp. under the tradename Polymer LM-200™. Other suitable cellulosic polymers include: quaternary nitrogen-containing cellulose ethers, such as those described in more detail in U.S. Pat. No. 3,962,418; and copolymers of etherified cellulose and starch, such as those described in more detail in U.S. Pat. No. 3,958,581.

Most preferably, the cellulosic polymer is carboxy methyl cellulose, typically having the following general formula:

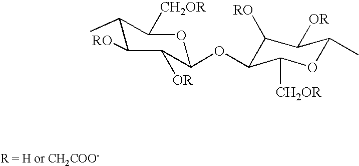

and wherein at least one R moiety is CH₂COO—.

### Adjunct Ingredients

Suitable adjunct materials include, but are not limited to, surfactants, builders, chelating agents, dye transfer inhibiting agents, dispersants, additional enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, solvents and/or pigments. In addition to the disclosure below, suitable examples of such other adjuncts and levels of use are found in U.S. Pat. Nos. 5,576,282, 6,306,812 and 6,326,348.

### EXAMPLES

### Examples 1-8

Liquid laundry detergent compositions suitable for front-loading automatic washing machines.

| | Composition | | | | | | | |
|---|---|---|---|---|---|---|---|---|
| | (wt % of composition) | | | | | | | |
| Ingredient | 1 | 2 | 3 | 4 | 5 | 6 | 7 | 8 |
| Alkylbenzene sulfonic acid | 7 | 11 | 4.5 | 1.2 | 1.5 | 12.5 | 5.2 | 4 |
| Sodium C₁₂₋₁₄ alkyl ethoxy 3 sulfate | 2.3 | 3.5 | 4.5 | 4.5 | 7 | 18 | 1.8 | 2 |
| C₁₄₋₁₅ alkyl 8-ethoxylate | 5 | 8 | 2.5 | 2.6 | 4.5 | 4 | 3.7 | 2 |
| C₁₂ alkyl dimethyl amine oxide | — | — | 0.2 | — | — | — | — | — |
| C₁₂₋₁₄ alkyl hydroxyethyl dimethyl | — | — | — | 0.5 | — | — | — | — |
| ammonium chloride | | | | | | | | |
| C₁₂₋₁₈ Fatty acid | 2.6 | 4 | 4 | 2.6 | 2.8 | 11 | 2.6 | 1.5 |
| Citric acid | 2.6 | 3 | 1.5 | 2 | 2.5 | 3.5 | 2.6 | 2 |
| Protease (Purafect ® Prime) | 0.5 | 0.7 | 0.6 | 0.3 | 0.5 | 2 | 0.5 | 0.6 |
| Amylase (Natalase ®) | 0.1 | 0.2 | 0.15 | — | 0.05 | 0.5 | 0.1 | 0.2 |
| Mannanase (Mannaway ®) | 0.05 | 0.1 | 0.05 | — | — | 0.1 | 0.04 | — |
| Xyloglucanase XYG1006* | 1 | 4 | 3 | 3 | 2 | 8 | 2.5 | 4 |
| (mg aep/100 g detergent) | | | | | | | | |
| Random graft co-polymer¹ | 1 | 0.2 | 1 | 0.4 | 0.5 | 2.7 | 0.3 | 1 |
| A compound having the following | 0.4 | 2 | 0.4 | 0.6 | 1.5 | 1.8 | 0.7 | 0.3 |
| general structure: | | | | | | | | |
| bis((C₂H₅O)(C₂H₄O)n)(CH₃)—N⁺—CₓH₂ₓ—N⁺—(CH₃)- | | | | | | | | |
| bis((C₂H₅O)(C₂H₄O)n), wherein n = | | | | | | | | |
| from 20 to 30, and x = from 3 to | | | | | | | | |
| 8, or sulphated or sulphonated | | | | | | | | |
| variants thereof | | | | | | | | |
| Ethoxylated Polyethylenimine² | — | — | — | — | — | 0.5 | — | — |
| Amphiphilic alkoxylated grease | 0.1 | 0.2 | 0.1 | 0.2 | 0.3 | 0.3 | 0.2 | 0.3 |
| cleaning polymer³ | | | | | | | | |
| Diethoxylated poly (1,2 propylene | — | — | — | — | — | — | 0.3 | — |
| terephthalate short block soil | | | | | | | | |
| release polymer. | | | | | | | | |
| Diethylenetriaminepenta(methylene | 0.2 | 0.3 | — | — | 0.2 | — | 0.2 | 0.3 |
| phosphonic) acid | | | | | | | | |
| Hydroxyethane diphosphonic acid | — | — | 0.45 | — | — | 1.5 | — | 0.1 |
| FWA | 0.1 | 0.2 | 0.1 | — | — | 0.2 | 0.05 | 0.1 |
| Solvents (1,2 propanediol, | 3 | 4 | 1.5 | 1.5 | 2 | 4.3 | 2 | 1.5 |
| ethanol), stabilizers | | | | | | | | |
| Hydrogenated castor oil derivative structurant | 0.4 | 0.4 | 0.3 | 0.1 | 0.3 | — | 0.4 | 0.5 |
| Boric acid | 1.5 | 2.5 | 2 | 1.5 | 1.5 | 0.5 | 1.5 | 1.5 |
| Na formate | — | — | — | 1 | — | — | — | — |
| Reversible protease inhibitor⁴ | — | — | 0.002 | — | — | — | — | — |
| Perfume | 0.5 | 0.7 | 0.5 | 0.5 | 0.8 | 1.5 | 0.5 | 0.8 |
| Perfume MicroCapsules slurry | 0.2 | 0.3 | 0.7 | 0.2 | 0.05 | 0.4 | 0.9 | 0.7 |
| (30% am) | | | | | | | | |
| Ethoxylated thiophene Hueing Dye | | | | | | | 0.007 | 0.008 |

| | |
|---|---|
| Buffers (sodium hydroxide, Monoethanolamine) | To pH 8.2 |
| Water and minors (antifoam, aesthetics) | To 100% |

### Examples 9-16

Liquid laundry detergent compositions suitable for top-loading automatic washing machines.

| | Composition | | | | | | | |
|---|---|---|---|---|---|---|---|---|
| | (wt % of composition) | | | | | | | |
| Ingredient | 9 | 10 | 11 | 12 | 13 | 14 | 15 | 16 |
| C₁₂₋₁₅ Alkylethoxy(1.8)sulfate | 20.1 | 15.1 | 20.0 | 15.1 | 13.7 | 16.7 | 10.0 | 9.9 |
| C_{11.8} Alkylbenzene sulfonate | 2.7 | 2.0 | 1.0 | 2.0 | 5.5 | 5.6 | 3.0 | 3.9 |
| C₁₆₋₁₇ Branched alkyl sulfate | 6.5 | 4.9 | | 4.9 | 3.0 | 9.0 | 2.0 | |
| C₁₂₋₁₄ Alkyl-9-ethoxylate | 0.8 | 0.8 | 0.8 | 0.8 | 8.0 | 1.5 | 0.3 | 11.5 |
| C₁₂ dimethylamine oxide | | | 0.9 | | | | | |
| Citric acid | 3.8 | 3.8 | 3.8 | 3.8 | 3.5 | 3.5 | 2.0 | 2.1 |
| C₁₂₋₁₈ fatty acid | 2.0 | 1.5 | 2.0 | 1.5 | 4.5 | 2.3 | | 0.9 |
| Protease (Purafect ® Prime) | 1.5 | 1.5 | 0.5 | 1.5 | 1.0 | 1.8 | 0.5 | 0.5 |
| Amylase (Natalase ®) | 0.3 | 0.3 | 0.3 | 0.3 | 0.2 | 0.4 | | |
| Amylase (Stainzyme ®) | | | | | | | | 1.1 |
| Mannanase (Mannaway ®) | 0.1 | | | | | 0.1 | | |
| Pectate Lyase (Pectawash ®) | 0.1 | | | | | 0.2 | | |
| Xyloglucanase XYG1006* | 5 | 13 | 2 | 5 | 20 | 1 | 2 | 3 |
| (mg aep/100 g detergent) | | | | | | | | |
| Borax | 3.0 | 3.0 | | | 2.0 | 3.0 | 3.0 | 3.3 |
| Na & Ca formate | 0.2 | 0.2 | | 0.2 | 0.2 | | 0.7 | |
| A compound having the | 1.6 | 1.6 | 3.0 | 1.6 | 2.0 | 1.6 | 1.3 | 1.2 |
| following general structure: | | | | | | | | |
| bis((C₂H₅O)(C₂H₄O)n)(CH₃)—N⁺—CₓH₂ₓ—N⁺—(CH₃)- | | | | | | | | |
| bis((C₂H₅O)(C₂H₄O)n), wherein n = from 20 to 30, | | | | | | | | |
| and x = from 3 to 8, or sulphated or sulphonated | | | | | | | | |
| variants thereof | | | | | | | | |
| Random graft co-polymer¹ | 0.4 | 0.2 | 1.0 | 0.5 | 0.6 | 1.0 | 0.8 | 1.0 |
| Diethylene triamine | 0.4 | 0.4 | 0.4 | 0.4 | 0.2 | 0.3 | 0.8 | |
| pentaacetic acid | | | | | | | | |
| Tinopal AMS-GX | 0.2 | 0.2 | 0.2 | 0.2 | 0.2 | 0.3 | 0.1 | |
| Tinopal CBS-X | | | | | | 0.1 | | 0.2 |
| Amphiphilic alkoxylated | 1.0 | 1.3 | 1.3 | 1.4 | 1.0 | 1.1 | 1.0 | 1.0 |
| grease cleaning polymer³ | | | | | | | | |
| Texcare 240N (Clariant) | | | | 1.0 | | | | |
| Ethanol | 2.6 | 2.6 | 2.6 | 2.6 | 1.8 | 3.0 | 1.3 | |
| Propylene Glycol | 4.6 | 4.6 | 4.6 | 4.6 | 3.0 | 4.0 | 2.5 | |
| Diethylene glycol | 3.0 | 3.0 | 3.0 | 3.0 | 3.0 | 2.7 | 3.6 | |
| Polyethylene glycol | 0.2 | 0.2 | 0.2 | 0.2 | 0.1 | 0.3 | 0.1 | 1.4 |
| Monoethanolamine | 2.7 | 2.7 | 2.7 | 2.7 | 4.7 | 3.3 | 1.7 | 0.4 |
| Triethanolamine | | | | | | | | 0.9 |
| NaOH | to pH | to pH | to pH | to pH | to pH | to pH | to pH | to pH |
| | 8.3 | 8.3 | 8.3 | 8.3 | 8.3 | 8.3 | 8.3 | 8.5 |
| Suds suppressor | | | | | | | | |
| Dye | 0.01 | 0.01 | 0.01 | | 0.01 | 0.01 | 0.01 | 0.0 |
| Perfume | 0.5 | 0.5 | 0.5 | 0.5 | 0.7 | 0.7 | 0.8 | 0.6 |
| Perfume MicroCapsules | 0.2 | 0.5 | 0.2 | 0.3 | 0.1 | 0.3 | 0.9 | 1.0 |
| slurry (30% am) | | | | | | | | |
| Ethoxylated thiophene | | | | | 0.002 | 0.004 | | |
| Hueing Dye | | | | | | | | |
| Water | balance | balance | balance | balance | balance | balance | balance | balance |

### Examples 17-22

The following are granular detergent compositions produced in accordance with the invention suitable for laundering fabrics.

| | 17 | 18 | 19 | 20 | 21 | 22 |
|---|---|---|---|---|---|---|
| Linear alkylbenzenesulfonate | 15 | 12 | 20 | 10 | 12 | 13 |
| with aliphatic carbon chain | | | | | | |
| length C₁₁-C₁₂ | | | | | | |
| Other surfactants | 1.6 | 1.2 | 1.9 | 3.2 | 0.5 | 1.2 |
| Phosphate builder(s) | 2 | 25 | 4 | 3 | 2 | |
| Zeolite | | 1 | | 1 | 4 | 1 |
| Silicate | 4 | 5 | 2 | 3 | 3 | 5 |
| Sodium Carbonate | 9 | 20 | 10 | 17 | 5 | 23 |
| Polyacrylate (MW 4500) | 1 | 0.6 | 1 | 1 | 1.5 | 1 |
| Carboxymethyl cellulose | 1 | — | 0.3 | — | 1.1 | — |
| (Finnfix BDA ex CPKelco) | | | | | | |
| Xyloglucanase XYG1006* | 1.5 | 2.4 | 1.7 | 0.9 | 5.3 | 2.3 |
| (mg aep/100 g detergent) | | | | | | |
| Other enzymes powders | 0.23 | 0.17 | 0.5 | 0.2 | 0.2 | 0.6 |
| Fluorescent Brightener(s) | 0.16 | 0.06 | 0.16 | 0.18 | 0.16 | 0.16 |
| Diethylenetriamine | 0.6 | | 0.6 | 0.25 | 0.6 | 0.6 |
| pentaacetic acid or Ethylene | | | | | | |
| diamine tetraacetic acid | | | | | | |
| MgSO₄ | 1 | 1 | 1 | 0.5 | 1 | 1 |
| Bleach(es) and Bleach | 6.88 | | 6.12 | 2.09 | 1.17 | 4.66 |
| activator(s) | | | | | | |

| | |
|---|---|
| Sulfate/Moisture/perfume | Balance to 100% |

### Examples 23-28

The following are granular detergent compositions produced in accordance with the invention suitable for laundering fabrics.

| | 23 | 24 | 25 | 26 | 27 | 28 |
|---|---|---|---|---|---|---|
| Linear alkylbenzenesulfonate | 8 | 7.1 | 7 | 6.5 | 7.5 | 7.5 |
| with aliphatic carbon chain | | | | | | |
| length C₁₁—C₁₂ | | | | | | |
| Other surfactants | 2.95 | 5.74 | 4.18 | 6.18 | 4 | 4 |
| Layered silicate | 2.0 | — | 2.0 | — | — | — |
| Zeolite | 7 | — | 2 | — | 2 | 2 |
| Citric Acid | 3 | 5 | 3 | 4 | 2.5 | 3 |
| Sodium Carbonate | 15 | 20 | 14 | 20 | 23 | 23 |
| Silicate | 0.8 | — | 0.11 | — | — | — |
| Soil release agent | 0.75 | 0.72 | 0.71 | 0.72 | — | — |
| Acrylic Acid/Maleic Acid | 1.1 | 3.7 | 1.0 | 3.7 | 2.6 | 3.8 |
| Copolymer | | | | | | |
| Carboxymethyl cellulose | 0.15 | — | 0.2 | — | 1 | — |
| (Finnfix BDA ex CPKelco) | | | | | | |
| Xyloglucanase XYG1006* | 3.1 | 2.34 | 3.12 | 4.68 | 3.52 | 7.52 |
| (mg aep/100 g detergent) | | | | | | |
| Other enzyme powders | 0.65 | 0.75 | 0.7 | 0.27 | 0.47 | 0.48 |
| Bleach(es) and bleach | 16.6 | 17.2 | 16.6 | 17.2 | 18.2 | 15.4 |
| activator(s) | | | | | | |

| | |
|---|---|
| Sulfate/Water & | Balance to 100% |
| Miscellaneous | |

¹ Random graft copolymer is a polyvinyl acetate grafted polyethylene
oxide copolymer having a polyethylene oxide backbone and multiple
polyvinyl acetate side chains. The molecular weight of the polyethylene
oxide backbone is about 6000 and the weight ratio of the polyethylene
oxide to polyvinyl acetate is about 40 to 60 and no more than 1 grafting
point per 50 ethylene oxide units.
² Polyethylenimine (MW = 600) with 20 ethoxylate groups per —NH.
³ Amphiphilic alkoxylated grease cleaning polymer is a polyethylenimine
(MW = 600) with 24 ethoxylate groups per —NH and 16 propoxylate
groups per —NH
⁴ Reversible Protease inhibitor of structure:

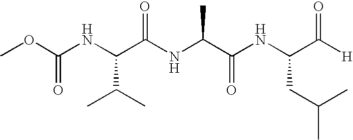

* Remark: all enzyme levels expressed as % enzyme raw material, except
for xyloglucanase where the level is given in mg active enzyme protein
per 100 g of detergent.
XYG1006 enzyme is according to SEQ ID: 1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Use of a cellulase to impart soil release benefits to cotton during a subsequent laundering process.

2. Use according to claim 1, wherein the cellulase is incorporated in a laundry detergent composition.

3. Use according to claim 2, wherein the laundry detergent composition additionally comprises a detersive surfactant.

4. Use according to claim 1, wherein the cellulase has endo beta 1,4-glucanase activity and a structure which does not comprise a class A carbohydrate binding module.

5. Use according to claim 1, wherein the cellulase is a glycosyl hydrolase having enzymatic activity towards amorphous cellulose substrates, wherein the glycosyl hydrolase is selected from GH families 5, 7, 12, 16, 44 or 74.

6. Use according to claim 1, wherein the cellulase is a glycosyl hydrolase having enzymatic activity towards both xyloglucan and amorphous cellulose substrates, wherein the glycosyl hydrolase is selected from GH families 5, 12, 44 or 74.

7. Use according to claim 1, wherein the cellulase modifies the fabric surface during the laundering process so as to improve the removal of soils adhered to the fabric after the laundering process during wearing and usage of the fabric, in subsequent wash cycles.

8. Use according to claim 7, wherein the cellulase modifies the fabric surface during the laundering process so as to improve the removal of soils adhered to the fabric after the laundering process during wearing and usage of the fabric, in the subsequent three wash cycles.

9. Use according to claim 1, wherein the cellulase is used at a concentration of 0.005 ppm to 1.0 ppm in the wash liquor during the laundering process.

10. Use according to claim 9, wherein the cellulase is used at a concentration of 0.02 ppm to 0.5 ppm in the wash liquor during the laundering process.

11. Use according to claim 1, wherein the cellulase is incorporated in a laundry detergent composition that additionally comprises a cellulosic polymer.

12. Use according to claim 1, wherein the cellulase is incorporated in a laundry detergent composition that additionally comprises a polyester soil release polymer.

13. Use according to claim 1, wherein the cellulase is incorporated in a laundry detergent composition, wherein the laundry detergent composition is at least partially enclosed by a water-soluble film.


SEQUENCE LISTING (1 sequence)

SEQ ID NO: 1 - protein, 524 aa, Paenibacillus polyxyma
VVHGQTAKTITIKVDTFKDRKPISPYIYGTNQDLAGDENMAARRLGGNRMTGYNWENNMSNAGSDWQQSSDNYLCSNGGLTQAECEKPGAVTTSFHDQSLKLGTYSLVTLPMAGYVAKDGNGSVQESEKAPSARWNQVVNAKNAPFQLQPDLNDNRVYVDEFVHFLVNKYGTASTKAGVKGYALDNEPALWSHTHPRIHGEKVGAKELVDRSVSLSKAVKAIDAGAEVFGPVLYGFGAYKDLQTAPDWDSVKGNYSWFVDYYLDQMRLSSQVEGKRLLDVFDVHWYPEAMGGGIRITNEVGNDETKKARMQAPRTLWDPTYKEDSWIAQWNSEFLPILPRLKQSVDKYYPGTKLAMTEYSYGGENDISGGIAMTDVLGILGKNDVYMANYWKLKDGVNNYVSAAYKLYRNYDGKNSTFGDTSVSAQTSDIVNSSVHASVTNASDKELHLVVMNKSMDSAFDAQFDLSGAKTYISGKVWGFDKNSSQIKEAAPITQISGNRFTYTVPPLTAYHIVLTTGNDTSPV